(19) United States
(12) Patent Application Publication
(10) Pub. No.: US 2015/0033820 A1
(43) Pub. Date: Feb. 5, 2015

(54) APPARATUS FOR INSPECTING PHYSICAL QUALITY SENSOR

(71) Applicant: SAMSUNG ELECTRO-MECHANICS CO., LTD., Suwon-Si (KR)

(72) Inventors: Hee Jung KANG, Suwon-Si (KR); Jeom Soo Cho, Suwon-Si (KR); Gi Suk Kim, Suwon-Si (KR)

(73) Assignee: SAMSUNG ELECTRO-MECHANICS CO., LTD., Suwon-Si (KR)

(21) Appl. No.: 14/249,861

(22) Filed: Apr. 10, 2014

(30) Foreign Application Priority Data
Aug. 2, 2013 (KR) 10-2013-0091829

(51) Int. Cl. G01K 15/00; G01N 33/00

(52) U.S. Cl. CPC G01K 15/007; G01N 33/0006
USPC 73/1.01

(57) ABSTRACT

Disclosed herein is an apparatus for inspecting a physical quality sensor, the apparatus including: a measuring unit measuring at least one reference sensor and an inspected sensor; and a display unit electrically communicated with the measuring unit. The measuring unit includes: a base including a first seating part mounting a reference substrate having at least one reference sensor mounted thereon and a second seating part seating the inspected sensor to be adjacent to the reference sensor; and an upper cover including a through-hole exposing the reference sensor and the inspected sensor to surrounding atmosphere and formed in a plate shape covering at least one reference sensor and the inspected sensor. According to the present invention, measured data of the inspected sensor is compared to measured data of the reference sensor under the same condition, such that it is determined that the inspected sensor is a good product.

### CROSS REFERENCE TO RELATED APPLICATION

This application claims the benefit of Korean Patent Application No. 10-2013-0091829, filed on Aug. 2, 2013, entitled “Apparatus for Inspecting Physical Quality Sensor”, which is hereby incorporated by reference in its entirety into this application.

### BACKGROUND OF THE INVENTION

1. Technical Field

The present invention relates to an apparatus for inspecting a physical quality sensor capable of providing high reliability about whether or not the physical quality sensor, for example, a temperature and/or a humidity sensor is a good product.

2. Description of the Related Art

A physical quality sensor such as a temperature sensor and/or a humidity sensor is recently embedded in a smartphone, a tablet PC, or the like to be utilized in various applications. The physical quality sensor such as the temperature sensor and/or the humidity sensor may measure surrounding environment or a state of related components in real time and apply the measured information collected as described above through an application.

The physical quality sensor is mounted in a flexible printed circuit board (FPCB), a printed circuit board (PCB), and the like and is embedded in the smartphone or the tablet PC in order to be utilized in various devices as is well-known to those skilled in the art.

Before the physical quality sensor is mounted, the physical quality sensor needs to be inspected about whether or not it has defects.

An apparatus for inspecting the sensor as mentioned above is disclosed in Patent Document 1, and the apparatus includes a sensor assembly having inspecting sensors arranged therein, a horizontal position controller capable of moving the sensor assembly in a horizontal direction, and a vertical position controller capable of moving the sensor assembly in a vertical direction, thereby making it possible to accurately match the inspecting sensors to detecting sensors.

The apparatus for inspecting the sensor according to the prior art may check only whether or not the inspecting sensors malfunction through the detecting sensors and have a complex mechanism for assisting location of the inspecting sensors.

However, the physical quality sensor such as the temperature sensor and/or the humidity sensor may not practically guarantee reliability of a sensing operation in addition to the malfunction inspection of the sensor. Therefore, a method capable of measuring precision and accuracy of the physical quality sensor should be considered.

### PRIOR ART DOCUMENT

### Patent Document

(Patent Document 1) Korea Patent Laid-Open Publication No. 10-2003-0039110

### SUMMARY OF THE INVENTION

The present invention has been made in an effort to provide an apparatus for inspecting a physical quality sensor capable of determining whether the physical quality sensor is a good product or a defective product.

According to a preferred embodiment of the present invention, there is provided an apparatus for inspecting a physical quality sensor of measuring physical quality by a reference sensor and an inspected sensor under the same condition to determine whether or not the inspected sensor is a good product, the apparatus including: a measuring unit measuring at least one reference sensor and the inspected sensor; and a display unit electrically communicated with the measuring unit.

The measuring unit may include: a base including a first seating part mounting a reference substrate having at least one reference sensor mounted thereon and a second seating part seating the inspected sensor to be adjacent to the reference sensor; and an upper cover including a through-hole exposing the reference sensor and the inspected sensor to surrounding atmosphere and formed in a plate shape covering at least one reference sensor and the inspected sensor.

The through-hole may be formed in a portion of the upper cover facing at least one reference sensor and the inspected sensor seated on the base.

The through-hole may communicate between an inner space defined by the base and the upper cover and surrounding environment to thereby provide a condition subjected to the surrounding environment.

The through-hole may be mounted with a blower so that heat generated at the time of a measurement is rapidly discharged to the surrounding environment (or the outside).

The blower may be installed on the base or the upper cover while not being limited to the through-hole to thereby blow air toward the through-hole.

The second seating part may have a printed circuit board (PCB) connector electrically contacting a terminal of an inspected PCB having the inspected sensor mounted thereon.

The upper cover may further include a pressurizing member on a lower surface facing the PCB connector, wherein the pressurizing member may pressurize the terminal of the inspected PCB to thereby guarantee good contact between the terminal of the inspected PCB and the PCB connector.

The pressurizing member may include an elastic piece.

The apparatus may further include a controlling unit operating measured data of at least one reference sensor and measured data of the inspected sensor and controlling the measuring unit and the display unit.

The upper cover may be hinge-coupled to the base and the base may further include a locker operating by an electromagnet principle.

The display unit and the measuring unit may be electrically communicated with each other instead of being separately separated from each other in order to perform an accurate measurement of the measuring unit.

The display unit may include a display window and a light bar.

At least one reference sensor and the inspected sensor may be disposed at the same height as each other and spaced apart from each other at equidistance so as to be measured under the same condition.

The first seating part may include a heat radiating plate allowing heat emitted from the reference substrate to be discharged.

The first seating part may install the reference to be attachable and detachable thereto and therefrom to thereby allow repair and replacement of the reference substrate.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a perspective view schematically showing an apparatus for inspecting a physical quality sensor according to a preferred embodiment of the present invention;

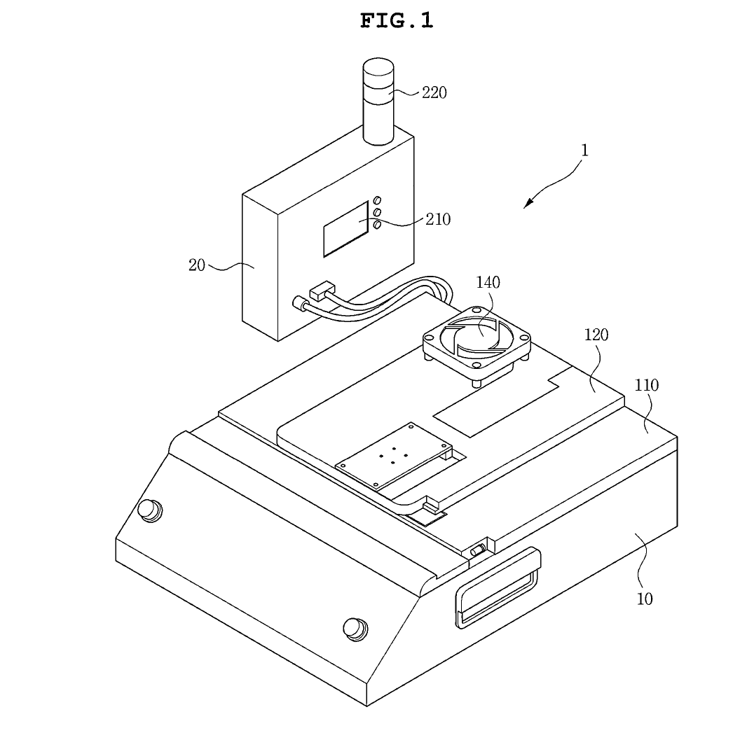

FIG. 2 is a perspective view schematically showing the apparatus for inspecting the physical quality sensor having an upper cover opened according to a preferred embodiment of the present invention;

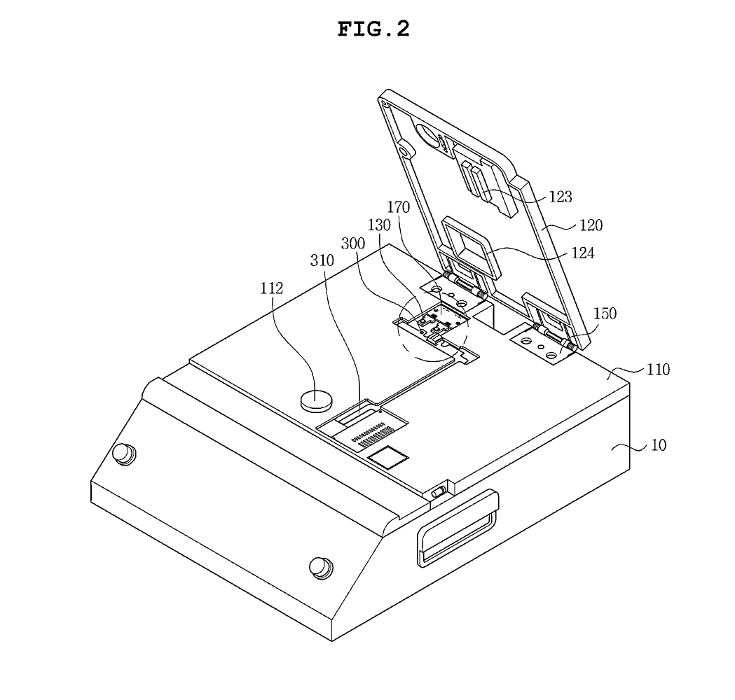

FIG. 3 is an exploded perspective view of the physical quality sensor shown in FIG. 1; and

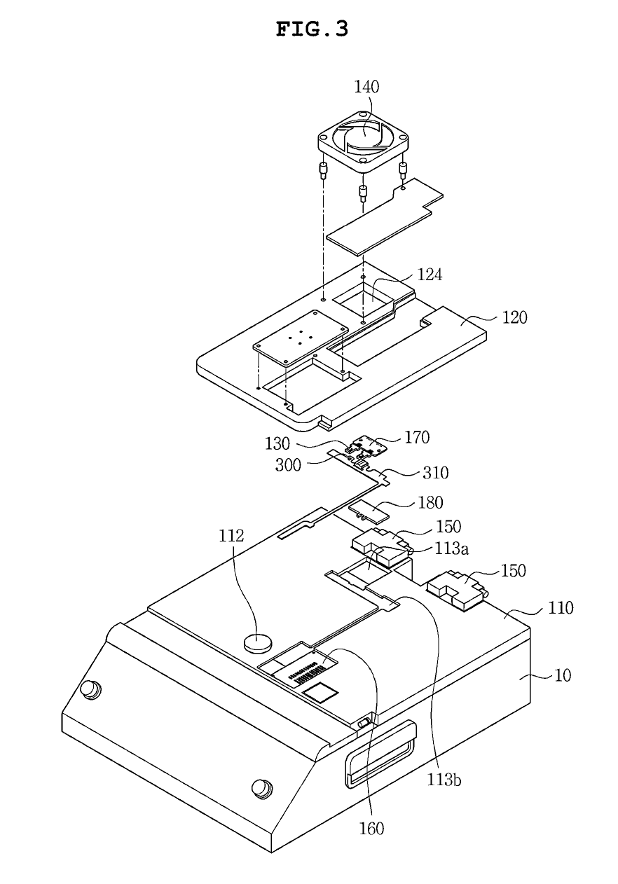

FIG. 4 is an enlarged view of an arc portion of FIG. 2.

### DESCRIPTION OF THE PREFERRED EMBODIMENTS

The objects, features and advantages of the present invention will be more clearly understood from the following detailed description of the preferred embodiments taken in conjunction with the accompanying drawings. Throughout the accompanying drawings, the same reference numerals are used to designate the same or similar components, and redundant descriptions thereof are omitted. Further, in the following description, the terms “first”, “second”, “one side”, “the other side” and the like are used to differentiate a certain component from other components, but the configuration of such components should not be construed to be limited by the terms. Further, in the description of the present invention, when it is determined that the detailed description of the related art would obscure the gist of the present invention, the description thereof will be omitted.

Hereinafter, preferred embodiments of the present invention will be described in detail with reference to the accompanying drawings.

An apparatus 1 for inspecting a physical quality sensor according to a preferred embodiment of the present invention is configured to include a measuring unit 10 and a display unit 20. The present invention may further include a controlling unit (not shown) controlling a sensing measurement of a reference sensor and an inspected sensor, an opening and closing of an upper cover, and the like, and an illustration and a description of an electrical wiring will be omitted in this specification in order to assist in clearly understanding the present invention. For reference, the controlling unit may be provided in the measuring unit 10. Unlike this, it may be connected to a separate exterior unit.

The physical quality sensor to be described in this specification is described based on a temperature sensor and/or a humidity sensor, but is not limited thereto. That is, the present invention may be applied to other types of sensors measuring physical quality such as a vapor sensor, an illuminance sensor, or the like. In other words, the present invention, which inspects whether or not the physical quality sensor is a good product, is designed to determine whether or not the inspected sensor is normal by referencing to the reference sensor.

The measuring unit 10 according to the preferred embodiment of the present invention includes a base 110 receiving the reference sensor and the inspected sensor, an upper cover 120 covering the reference sensor and the inspected sensor, and a blower 140 disposed on the upper cover 120.

In addition, the apparatus 1 for inspecting the physical quality sensor includes a display unit 20 having a display window 210 displaying measured data of the reference sensor mounted on the measuring unit 10 and measured data of the inspected sensor to be seated on the measuring unit 10 and a light bar 220 capable of confirming whether or not the inspected sensor is defective.

The display unit 20 includes the light bar 220 capable of warning the defect of the inspected sensor 300 when the measured data of the inspected sensor 300 is out of an allowable error range of (average) measured data of the reference sensor 130 by comparison between the measured data of the reference sensor and the measured data of the inspected sensor using the controlling unit. The display unit 20 is provided separately from the measuring unit 10 except for electrical communication through wiring as shown in the drawings. This is to minimize an effect of factors such as heat generated from the display window 210 and the light bar 220 of the display unit 20, and the like on the reference sensor 130 and the inspected sensor 300 mounted on the measuring unit 10.

The display unit 20 is provided with the display window 210 as described above. Specifically, the display window 210 enables a worker to directly confirm the measured data of the respective reference sensors, average measured data thereof, the measured data of the inspected sensor, whether or not the inspected sensor is defective, or the like, by the naked eye.

The apparatus 1 for inspecting the physical quality sensor according to the preferred embodiment of the present invention concavely forms seating parts 113a and 113b separately receiving the reference sensor 130 and the inspected sensor 300, on the base 110. A first seating part 113a is a space in which at least one reference sensor 130 is mounted together with a reference substrate 170 electrically communicated with the controlling unit, and a second seating part 113b is disposed to be adjacent to the first seating part 113a and is formed to correspond to a size and shape of an inspected printed circuit board (PCB) 310 so that it may smoothly receive the inspected PCB 310 having the inspected sensor 300 mounted thereon. Here, the reference sensor 130 normally measures the physical quality (temperature, humidity, or the like) of surrounding atmosphere to thereby provide a reference of determining a defective product and/or a good product. Therefore, the reference sensor 130 and the inspected sensor 300 need to be disposed to adjacent to each other in order to measure the physical quality such as the temperature and/or humidity under the same condition, and at least one reference sensor 130, preferably, two reference sensors 130 are provided in order to increase reliability of the measured data of the reference sensor.

Further, according to the preferred embodiment of the present invention, the first seating part 113a may be further provided with a heat radiating plate 180. The heat radiating plate 180 is disposed on a lower portion of the reference substrate 170, and specifically, is interposed between the reference substrate 170 and the first seating part 113a, thereby making it possible to additionally radiate the heat generated from the reference substrate 170 at the time of the measurement.

Alternatively, according to the preferred embodiment of the present invention, the reference substrate 170 having at least one reference sensor 130 mounted thereon is mounted to be attachable to/detachable from the first seating part 113a to thereby easily perform repair and replacement at the time of the malfunction of the reference sensor 130.

According to the preferred embodiment of the present invention, the physical quality (e.g., the temperature, the humidity, or the like) of the surrounding atmosphere is measured by at least one reference sensor 130 and the physical quality of the same surrounding atmosphere is measured by the inspected sensor 300. Typically, even in the case in which a surrounding environment and a mounting condition are changed, since the temperature and humidity indicate a significant difference, the apparatus for inspecting the physical quality sensor according to the preferred embodiment of the present invention needs to be designed to measure the reference sensor 130 and the inspected sensor 300 seated on the base 110 under a condition as equal as possible.

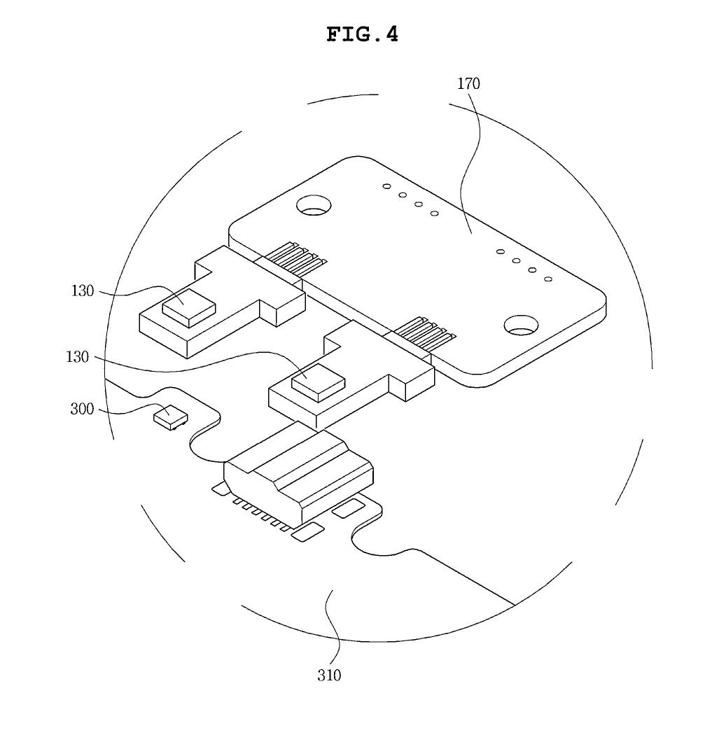

The inspected sensor 300 is disposed at a middle point of at least one reference sensor 130, particularly two reference sensors 130 to be spaced apart from each other at equidistance (see an arc portion of FIG. 4). Unlike this, the inspected sensor 300 and the two reference sensors 130 may be arranged in a regular triangle by allowing gaps between the respective sensors 130 and 300 to be equal while forming the respective vertexes. In addition, the inspected sensor 300 may have the same set height as that of at least one reference sensor 130. In addition, according to the preferred embodiment of the present invention, the reference sensor 130 and the inspected sensor 300 are seated in an inner space defined by the base 110 and the upper cover 120, such that measuring environment is formed to be equal, thereby making it possible to more accurately perform the inspection.

As described above, the apparatus 1 for inspecting the physical quality sensor according to the preferred embodiment of the present invention may collect the measured data of the temperature and humidity measured by at least one reference sensor 130 and the inspected sensor 300 seated on the measuring unit 10 and may operate and store the measured data by the controlling unit. When the measured data of the inspected sensor 300 is within the allowable error range of the measured data measured by at least one reference sensor 130, it is determined that this inspected sensor is the good product, and when the measured date of the inspected sensor 300 is out of the allowable error range, it is determined that this inspected sensor 300 is defective. For reference, the respective measured data collected from at least one reference sensor 130 may be calculated as an average value to thereby be compared to the measured data of the inspected sensor 300.

The upper cover 120 is positioned on an upper portion of the base 110 to shield the seating parts 113a and 113b. Preferably, the upper cover 120 is elastically biased so as to be elastically supported on the upper portion of the base 110 via hinge parts 150. The seating parts 113a and 113b are concavely stepped downwardly based on the upper portion of the base 110 as shown in the drawing, such that the seating parts 113a and 113b may be partitioned from the surrounding environment only by arranging the upper cover 120 on the upper portion of the base 110 in parallel with each other. If necessary, side walls may be provided along edges of the upper cover 120. The above-mentioned side wall may partition the seating parts 113a and 113b from the surrounding environment even in the case in which the edge of the upper cover 120 and the upper portion of the base 110 are only in contact with each other, where the side wall serves as a spacer, thereby making it possible to space between a flat surface of the upper cover 120 and an upper surface of the base 110.

Additionally, the upper cover 120 is fixedly positioned at the upper portion of base 110 by driving a locker 112 provided on the upper portion of the base 110. The locker 112 is designed by an electromagnet. According to an electromagnet principle, when a current is applied, the locker 112 forms magnetic field and pulls the upper cover 120 by magnetic force to maintain the upper cover 120 in a closed state, while when the application of the current is stopped, the magnetic force becomes extinct, thereby forcedly opening the upper cover 120 via the hinge parts 150. The upper cover 120 may be made of a metal material so as to be opened and closed depending on an operation of the electromagnet of the locker 112.

Since the base 110 forms the second seating part 113b receiving the inspected PCB 310 having the inspected sensor 300 mounted thereon as described above, the inspected PCB 310 may be electrically connected by being simply put on the second seating part 113b concavely formed in the upper surface of the base 110. The worker is only required to put the inspected PCB 310 which is an object to be inspected on the second seating part 113b and close the upper cover 120 each time, such that the number of working processes is decreased and behaviors unnecessary for the operation of the apparatus 1 for inspecting the physical quality sensor at the time of the measurement are decreased, thereby making it possible to decrease malfunction cases.

The second seating part 113b has a PCB connector 160 provided to one side thereof The PCB connector 160 may be arranged with a terminal of the inspected PCB 310 in a vertical direction to thereby supply power required at the time of the measurement and collect the measured data of the inspected sensor 300.

Particularly, according to the preferred embodiment of the present invention, the upper cover 120 has a pressurizing member 123 provided on a lower surface thereof so that a contact state between the terminal of the inspected PCB 310 and the PCB connector 160 may be guaranteed. The pressurizing member 123 pressurizes the terminal of the inspected PCB 310 and the PCB connector 160 at the time of sealing the upper cover 120 to thereby closely adhere to each other, thereby assisting in electrically connecting between the terminal of the inspected PCB 310 and the PCB connector 160 to each other to be reliable. Alternatively, the pressurizing member 123 may be mounted with an elastic piece such as a spring to thereby assist in closely adhering the inspected PCB 310 and the PCB connector 160 to each other as described above by elastic and repellent force of the elastic piece and to serve as a buffer material by compressive force of the elastic piece when abnormal pressure is applied to the inspected PCB 310 and the PCB connector 160. Further, the pressurizing member 123, which is a member constantly positioning the inspected PCB 310 in the second seating part 113b, may apply power by being in contact with the terminal of the inspected PCB 310, if necessary.

To this end, the pressurizing member 123 is protruded vertically and downwardly from the lower surface of the upper cover 120 so as to face the PCB connector 160.

As shown, the upper cover 120 has a through-hole 124 formed therein. The through-hole 124 allows the inner space formed by the upper cover 120 and the upper surface of the base 110 to be exposed to the surrounding environment and allows the inner space between the upper surface of the base 110 and the upper cover 120 to be equal to the surrounding environment. In addition, the through-hole 124 is formed by punching one surface of the upper cover 120 facing at least one reference sensor 130 and the inspected sensor 300 so that the heat generated at the time of measurement of the reference sensor 130 and the inspected sensor 300 may be rapidly radiated to the outside. Preferably, the through-hole 124 of the upper cover 120 includes a blower 140. The blower 140 may rapidly radiate the heat in the inner space to the outside to thereby increase accuracy of the sensing operation and to form a condition similar to the surrounding atmosphere. The blower 140 may be easily attachable to/detachable from the through-hole 124 of the upper cover 120. The blower is not limited thereto, and is arranged to be perpendicular to the base 110 or the upper cover 120 so as to be adjacent to the through-hole 124, thereby making it possible to blow air toward the through-hole.

During a sensing process by the apparatus for inspecting the physical quality sensor, when the inspected sensor 300 is determined as a bad product in the case in which the measured data of the inspected sensor 300 is out of the allowable error range of the measured data of the reference sensor 130, an alarm function for the bad inspected sensor is provided by the display window 210 and/or the light bar 220 of the display unit 20 as described above, and the locker 112 is automatically set to be locked, such that the upper cover 120 may shield the base 110 to thereby retain the inspected sensor 300 on the second seating part 113b until a separate action is performed. Since all of these operations control the measuring unit 10 and the display unit 20 by the controlling unit, the worker is not required to perform other operations except for simply seating the inspected sensor 300 on the second seating part 113b and covering the upper cover 120. According to the preferred embodiment of the present invention, a chance of the malfunction by the worker at the time of the sensing operation of the inspected sensor by the apparatus for inspecting the physical quality sensor is minimized, thereby making it possible to more accurately and precisely determine whether or not the inspected sensor is the good product.

According to the preferred embodiment of the present invention, the measurement state of the humidity, the temperature, or the like of the inspected sensor may be determined with reference to the measurement information of the reference sensor.

According to the preferred embodiment of the present invention, the heat generated at the time of the operation of the inspecting apparatus is rapidly emitted and the atmosphere is formed so as to be maximally equal to the surrounding atmosphere, such that the error of the inspected sensor is minimized, thereby making it possible to improve reliability of the inspection.

According to the preferred embodiment of the present invention, it may be determined whether or not the inspected sensor malfunctions and it may also be determined whether or not the error in the sensing operation is present.

In addition, according to the preferred embodiment of the present invention, the fool proof capable of preventing the malfunction of the worker for the inspecting apparatus is configured when determining whether or not the inspected sensor is the good product, thereby making it possible to more precisely and accurately detect whether or not the sensing operation of the inspected sensor is performed.

Although the embodiments of the present invention have been disclosed for illustrative purposes, it will be appreciated that the present invention is not limited thereto, and those skilled in the art will appreciate that various modifications, additions and substitutions are possible, without departing from the scope and spirit of the invention.

Accordingly, any and all modifications, variations or equivalent arrangements should be considered to be within the scope of the invention, and the detailed scope of the invention will be disclosed by the accompanying claims.

What is claimed is:

1. An apparatus for inspecting a physical quality sensor, the apparatus comprising:
a measuring unit measuring at least one reference sensor and an inspected sensor; and
a display unit electrically communicated with the measuring unit,
wherein the measuring unit includes:
a base including a first seating part mounting a reference substrate having at least one reference sensor mounted thereon and a second seating part seating the inspected sensor to be adjacent to the reference sensor; and
an upper cover including a through-hole exposing the reference sensor and the inspected sensor to surrounding atmosphere and formed in a plate shape covering at least one reference sensor and the inspected sensor.

2. The apparatus as set forth in claim 1, wherein the through-hole is formed in the upper cover facing at least one reference sensor and the inspected sensor seated on the base.

3. The apparatus as set forth in claim 1, wherein the through-hole has a blower mounted thereon.

4. The apparatus as set forth in claim 1, wherein the second seating part has a printed circuit board (PCB) connector contacting a terminal of an inspected PCB having the inspected sensor mounted thereon.

5. The apparatus as set forth in claim 4, wherein the upper cover further includes a pressurizing member on a lower surface facing the PCB connector.

6. The apparatus as set forth in claim 5, wherein the pressurizing member includes an elastic piece.

7. The apparatus as set forth in claim 1, further comprising a controlling unit operating measured data of at least one reference sensor and measured data of the inspected sensor and controlling the measuring unit and the display unit.

8. The apparatus as set forth in claim 1, wherein the upper cover is hinge-coupled to the base.

9. The apparatus as set forth in claim 1, wherein the base further includes a locker operating by an electromagnet principle.

10. The apparatus as set forth in claim 1, wherein the display unit is separated from the measuring unit.

11. The apparatus as set forth in claim 1, wherein the display unit includes a display window and a light bar.

12. The apparatus as set forth in claim 1, wherein at least one reference sensor is disposed at the same height as the inspected sensor.

13. The apparatus as set forth in claim 1, wherein the inspected sensor and at least one reference sensor are spaced apart from each other at equidistance.

14. The apparatus as set forth in claim 1, wherein the first seating part includes a heat radiating plate.

15. The apparatus as set forth in claim 1, wherein the first seating part allows the reference substrate having at least one reference sensor mounted thereon to be attachable and detachable.

16. The apparatus as set forth in claim 1, further comprising a blower in the base or the upper cover.
